(19) United States
(12) Patent Application Publication
(10) Pub. No.: US 2023/0346677 A1
(43) Pub. Date: Nov. 2, 2023

(54) TOPICAL COMPOSITIONS CONTAINING VITAMIN C

(71) Applicant: Johnson & Johnson Consumer Inc., Skillman, NJ (US)

(72) Inventors: Shailendra Singh, Monroe Township, NJ (US); Star Marie Walsh, Upper Black Eddy, PA (US)

(21) Appl. No.: 18/139,463

(22) Filed: Apr. 26, 2023

Related U.S. Application Data
(60) Provisional application No. 63/335,875, filed on Apr. 28, 2022.

(51) Int. Cl. A61K 8/67; A61K 8/49; A61K 8/23; A61K 8/73; A61Q 19/00

(52) U.S. Cl. CPC A61K 8/676; A61K 8/4926; A61K 8/23; A61K 8/735; A61Q 19/00; A61K 2800/10

(57) ABSTRACT

The present invention provides topical compositions comprising a combination ascorbic acid, caffeine, an inorganic salt, and water that are single aqueous phase compositions.

### CROSS-REFERENCE TO RELATED APPLICATIONS

This application claims the benefit of U.S. provisional application 63/335,875 filed on Apr. 28, 2022, the complete disclosure of which is hereby incorporated herein by reference for all purposes.

### FIELD OF THE INVENTION

The present invention provides topical compositions comprising a combination ascorbic acid, caffeine, an inorganic salt, and water that are single phase aqueous compositions.

### BACKGROUND OF THE INVENTION

Ascorbic acid (Vitamin C) is widely used in skincare for its antioxidant and skin brightening properties. Typically, anhydrous formulations are employed since the stability of ascorbic acid in an aqueous vehicle is known to be challenging as water increases the oxidation kinetics for ascorbic acid.

Caffeine is also known to provide certain skincare benefits when used topically.

Applicants have now discovered improved topical compositions that are single phase aqueous compositions, preferably in the form of serums, containing high levels of ascorbic acid and caffeine. These compositions advantageously provide synergistic antioxidant activity and excellent anti-aging and skin brightening benefits. They also contain an inorganic salt such as sodium bisulfite and are highly stable.

### SUMMARY OF THE INVENTION

The present invention provides a topical composition comprising about 5% to about 22% by weight of ascorbic acid based on the total weight of the composition, about 1 to about 5% by weight of caffeine based on the total weight of the composition, at least 0.2% by weight of an inorganic salt selected from the group consisting of sodium sulfite, potassium sulfite, sodium bisulfite, and sodium metabisulfite based on the total weight of the composition, and at least 28% by weight of water based on the total weight of the composition, wherein the composition is a single aqueous phase.

The present invention also provides a topical composition comprising about 10% by weight of ascorbic acid based on the total weight of the composition, about 2% by weight of caffeine based on the total weight of the composition, about 0.5% by weight of sodium bisulfite based on the total weight of the composition, at least 38% by weight of water based on the total weight of the composition, about 40% by weight 1,3-propanediol based on the total weight of the composition, and about 0.2% by weight of ethylenediaminetetraacetic acid based on the total weight of the composition, wherein the composition is a single aqueous phase.

The present invention also provides a topical composition comprising about 15% by weight of ascorbic acid based on the total weight of the composition, about 2% by weight of caffeine based on the total weight of the composition, about 0.5% by weight of sodium bisulfite based on the total weight of the composition, at least 33% by weight of water based on the total weight of the composition, about 45% by weight 1,3-propanediol based on the total weight of the composition, and about 0.2% by weight of ethylenediaminetetraacetic acid based on the total weight of the composition.

The present invention also provides a topical composition comprising about 20% by weight of ascorbic acid based on the total weight of the composition, about 2% by weight of caffeine based on the total weight of the composition, at least 0.5% by weight of sodium bisulfite based on the total weight of the composition, at least 28% by weight of water based on the total weight of the composition, about 45% by weight 1,3-propanediol based on the total weight of the composition, and about 0.2% by weight of ethylenediaminetetraacetic acid based on the total weight of the composition.

The present invention further provides cosmetic methods for treating skin, comprising topically applying to skin in need of treatment for skin brightening or lightening these topical compositions.

### DETAILED DESCRIPTION OF THE INVENTION

Unless defined otherwise, all technical and scientific terms used herein have the meaning commonly understood by one of ordinary skill in the art to which the invention pertains. All publications, patent applications, patents, and other references mentioned herein are incorporated by reference.

As used herein, “topically applying” means directly laying on or spreading on outer skin, the scalp, or hair, e.g., by use of the hands or an applicator such as a wipe, roller, or spray.

As used herein, “cosmetically acceptable” means the ingredients the term describes are suitable for use in contact with tissues (e.g., the skin or hair) without undue toxicity, incompatibility, instability, irritation, allergic response, or the like.

As used herein, a “cosmetically acceptable active agent” is a compound (synthetic or natural) that has a cosmetic or therapeutic effect on the skin or hair.

Compositions of the present invention are suitable for treating signs of skin aging. As used herein, “signs of skin aging” includes the presence of lines including fine lines and wrinkles, loss of elasticity, uneven skin, blotchiness, diminished skin thickness, and abnormal or diminished synthesis of collagen, glycosaminoglycans, proteoglycans, elastin, or glycoproteins including fibronectin. In one embodiment, the sign of aging is selected from the presence of lines, fine lines, wrinkles, loss of elasticity, and abnormal or diminished synthesis of collagen, glycosaminoglycans, proteoglycans, elastin, or glycoproteins including fibronectin.

Compositions of the invention are also suitable for treating skin in need of brightening or lightening treatment. As used herein, the terms “brightening” or “lightening” the skin refers generally to lightening, brightening, whitening, and/or evening of the skin tone, skin color, and/or shade of skin, and/or to the reduction in sallowness, and/or to the lightening and/or fading of hyperpigmented marks and/or lesions including, but not limited to, pigmented spots, melanin spots, age spots, sun spots, senile lentigos, freckles, lentigos simplex, pigmented solar keratosis, seborrhoeic keratosis, melasma, acne marks, post-inflammatory hyperpigmentation, lentigines, ephelides, combinations of two or more thereof and the like. “Brightening” or “lightening” the skin also refers to increased skin radiance, glow, translucency and/or luminescence and/or obtaining a more radiant, glowing, translucent or luminous skin tone appearance or a less yellow or sallow skin tone. In certain preferred embodiments, “brightening” or “lightening” the skin refers to lightening and evening the skin tone, increasing skin radiance and/or lightening age spots. In certain other preferred embodiments, the present invention is directed to compositions and methods for use on skin in need of skin brightening or lightening treatment selected from sallow and/or darkened skin. In certain other preferred embodiments, the present invention is directed to compositions and methods for use on skin in need of skin brightening or lightening treatment selected from the group consisting of age spots, freckles, marks left after acne, and combinations of two or more thereof.

As used herein, “treatment” or “treating” means the amelioration, prophylaxis, or reversal of a condition, disease, or disorder, or at least one discernible symptom thereof. In one embodiment, “treatment” or “treating” refers to an amelioration, prophylaxis, or reversal of at least one measurable physical parameter related to the condition, disease, or disorder being treated, not necessarily discernible in or by the subject being treated. In another embodiment, “treatment” or “treating” refers to inhibiting or slowing the progression of a condition, disease, or disorder, either physically, e.g., stabilization of a discernible symptom, physiologically, e.g., stabilization of a physical parameter, or both. In another embodiment, “treatment” or “treating” refers to delaying the onset of a condition, disease, or disorder.

In certain embodiments, a composition of the invention is administered as a preventative measure. As used herein, “prevention” or “preventing” refers to a reduction of the risk of acquiring a given condition, disease, or disorder.

More broadly, the compositions of the invention may also be used to treat or prevent cosmetic, dermatological, or other conditions and disorders including, but not limited to, infections, deranged or disordered cutaneous or mucocutaneous tissue relevant to skin, nail and hair; oral, vaginal and anal mucosa; disturbed keratinization; inflammation; changes associated with intrinsic and extrinsic aging, and others which may or may not be related to cutaneous system. The manifestations include, but are not limited to, oily skin; acne; rosacea; age spots; blemished skin; blotches; cellulite; dermatoses; dermatitis; skin, nail and hair infections; dandruff; dryness or looseness of skin, nail and hair; xerosis; inflammation, or eczema; elastosis; herpes; hyperkeratosis; hyperpigmented skin; ichthyosis; keratoses; lentigines; melasmas; mottled skin; pseudofolliculitis barbae; photoaging and photodamage; pruritus; psoriasis; skin lines; stretch marks; thinning of skin, nail plate and hair; warts; wrinkles; oral or gum disease; irritated, inflamed, red, unhealthy, damaged or abnormal mucosa, skin, hair, nail, nostril, ear canal, anal or vaginal conditions; breakdown, defective synthesis or repair of dermal components; abnormal or diminished synthesis of collagen, glycosaminoglycans, proteoglycans and elastin, as well as diminished levels of such components in the dermis; uneven skin tone; uneven and rough surface of skin, nail and hair; loss or reduction of skin, nail and hair resiliency, elasticity and recoilability; laxity; lack of skin, nail and hair lubricants and luster; fragility and splitting of nail and hair; yellowing skin; reactive, irritating or telangiectatic skin; and dull and older-looking skin, nail and hair. In addition, the compositions of the current invention can be used for general care of skin, nail and hair; to improve skin texture and pores, flakiness and redness; to make skin soft, smooth, fresh, balanced, visibly clear, even-toned and brighter; to increase skin fullness and plumpness; and for skin bleach and lightening and wound healing; to reduce or prevent sweating or perspiration of underarm, crotch, palm, or other parts of the body.

As used herein, the term “subject” means any animal, preferably a mammal, most preferably a human, to whom a composition of the invention will be or has been administered. The term “mammal” as used herein, encompasses any mammal.

Examples of mammals include, but are not limited to, cows, horses, sheep, pigs, cats, dogs, mice, rats, rabbits, guinea pigs, monkeys, and humans. In a preferred embodiment, the subject is a human.

As used herein, “wrinkle” includes fine lines, fine wrinkles, or coarse wrinkles. Examples of wrinkles include, but are not limited to, fine lines around the eyes (e.g., “crow's feet”), forehead and cheek wrinkles, frown-lines, and laugh-lines around the mouth.

As used herein, “loss of elasticity” includes loss of elasticity or structural integrity of the skin or tissue, including but not limited to sagging, lax and loose tissue. The loss of elasticity or tissue structure integrity may be a result of a number of factors, including but not limited to disease, aging, hormonal changes, mechanical trauma, environmental damage, or the result of an application of products, such as a cosmetics or pharmaceuticals, to the tissue.

As used herein, “uneven skin” means a condition of the skin associated with diffuse or mottled pigmentation, which may be classified as hyperpigmentation, such as post-inflammatory hyperpigmentation.

As used herein, “blotchiness” means a condition of the skin associated with redness or erythema.

As used herein, “cosmetic” refers to beautifying or preserving, restoring, bestowing, simulating, or enhancing the appearance of bodily beauty or appearing to enhance beauty or youthfulness, specifically as it relates to the appearance of tissue or skin.

As used herein, “cosmetically effective amount” means an amount sufficient for treating or preventing one or more signs of skin aging, but low enough to avoid serious side effects. The cosmetically effective amount of the compound or composition will vary with the particular condition being treated, the age and physical condition of the end user, the severity of the condition being treated/prevented, the duration of the treatment, the nature of other treatments, the specific compound or product/composition employed, the particular cosmetically-acceptable carrier utilized, and like factors.

As used herein, “substantially free of” means the ingredient referred to is not directly or intentionally added to the formula. Preferably, “substantially free of” means containing less than about 1% of an ingredient. More preferably “substantially free of” means containing less than about 0.5% of an ingredient. Even more preferably “substantially free of” means containing less than about 0.1% by weight of an ingredient. The present compositions may be completely free of an ingredient, i.e., contain none of the ingredient.

Unless otherwise indicated, a percentage or concentration refers to a percentage or concentration by weight (i.e., % (W/W). A weight percent of an ingredient is based on the total weight of the composition containing the ingredient. Unless stated otherwise, all ranges are inclusive of the endpoints, e.g., “from 4 to 9” includes the endpoints 4 and 9.

### Ascorbic Acid

The composition comprises ascorbic acid, also known as Vitamin C Ascorbic acid has the following structure:

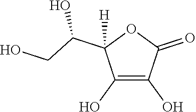 [SMILES: O=C1O[C@H]([C@@H](O)CO)C(O)=C1O]

The composition may contain about 5 to about 22% by weight of ascorbic acid based on the total weight of the composition. The composition may contain about 8 to about 12% by weight of ascorbic acid based on the total weight of the composition. The composition may contain about 13 to about 17% by weight of ascorbic acid based on the total weight of the composition. The composition may contain about 18 to about 22% by weight of ascorbic acid based on the total weight of the composition. The composition may contain about 10% by weight of ascorbic acid based on the total weight of the composition. The composition may contain about 15% by weight of ascorbic acid based on the total weight of the composition. The composition may contain about 20% by weight of ascorbic acid based on the total weight of the composition.

The composition may contain an unexpectedly high level of Vitamin C that remains stable over time. As known in the art, compositions containing Vitamin C are preferably prepared with an overage of the ingredient to account for some degradation during the product's shelf life.

### Caffeine

The composition also contains caffeine. The amount of caffeine in the composition may range from about 1 to about 5% by weight based on the total weight of the composition.

Preferably, the composition contains greater than 1% caffeine based on the total weight of the composition. More preferably, the composition contains about 2% by weight of caffeine based on the total weight of the composition.

### Inorganic Salt

The composition also contains an inorganic salt selected from the group consisting of sodium sulfite, potassium sulfite, sodium bisulfite, and sodium metabisulfite. Preferably, the inorganic salt is sodium bisulfite or sodium metabisulfite or a combination of the two. More preferably the inorganic salt is sodium bisulfite.

The amount of inorganic salt in the composition is at least about 0.2% by weight based on the total weight of the composition.

Preferably, the composition contains about 0.5% by weight of inorganic salt based on the total weight of the composition.

More preferably, the composition contains about 0.5% by weight of sodium bisulfite based on the total weight of the composition.

### Aqueous Vehicle

The composition is a single-phase aqueous solution. It comprises at least about 28% by weight of water based on the total weight of the composition. For example, the composition may comprise at least about 33% by weight water based on the total weight of the composition. The composition may comprise at least about 38% by weight water based on the total weight of the composition.

The composition may also contain 1,3-propanediol. The amount of 1,3-propanediol in the composition is preferably at least about 35% by weight of the composition. More preferably, the amount of 1,3-propanediol in the composition is at least 40% by weight of the composition. For example, the composition may contain about 45% by weight of 1,3-propanediol based on the total weight of the composition.

The composition may also contain ethylenediaminetetraacetic acid (EDTA). The amount of EDTA in the composition is preferably 0.1% by weight of water based on the total weight of the composition. For example, the composition may contain about 0.2% by weight of EDTA based on the total weight of the composition.

### Other Cosmetically Acceptable Active Agents

The composition may contain one or more other cosmetically acceptable active agents.

Cosmetically acceptable active agents include for example other anti-aging agents, other antioxidants, anti-acne agents, shine control agents, anti-microbial agents, anti-inflammatory agents, anti-mycotic agents, anti-parasite agents, external analgesics, sunscreens, photoprotectors, keratolytic agents, surfactants, moisturizers, nutrients, other vitamins, energy enhancers, anti-perspiration agents, astringents, deodorants, firming agents, anti-callous agents, and agents for hair and/or skin conditioning

The amount of other cosmetically active agent in the composition may range from about 0.001% to about 20% by weight of the composition, e.g., about 0.005% to about 10% by weight of the composition, such as about 0.01% to about 5% by weight of the composition, based on the total weight of the composition.

The cosmetically acceptable active agent may be selected for instance from alpha and polyhydroxy acids such as glycolic acid, lactic acid, malic acid, salicylic acid, citric acid, tartaric acid, and gluconolactone, benzoyl peroxide, D-panthenol carotenoids, retinoids such as retinol and retinyl palmitate, ceramides, polyunsaturated fatty acids, essential fatty acids, enzymes such as laccase, enzyme inhibitors, minerals, hormones such as estrogens, steroids such as hydrocortisone, 2-dimethylaminoethanol, copper salts such as copper chloride, peptides such as dipeptides including N-acyl dipeptide derivatives , tripeptides, argireline and syn-ake, those containing copper, coenzyme Q10, amino acids such as proline, other vitamins, lactobionic acid, acetyl-coenzyme A, niacin, riboflavin, thiamin, ribose, electron transporters such as NADH and FADH2, natural extracts such as those from Moringa oleifera seed, aloe vera, feverfew (for example, Chrysanthemum parthenium (Feverfew) Flower/Leaf/Stem Juice), oatmeal, dill, blackberry, princess tree, lemon aspen, resorcinols such as 4-hexyl resorcinol, curcuminoids, sugar amines such as N-acetyl glucosamine, glycosaminoglycans such as hyaluronic acid and chondroitin sulfate, and derivatives and mixtures thereof.

Examples of other vitamins include, but are not limited to, vitamin A, vitamin B′s such as vitamin B3, vitamin B5, and vitamin B12, vitamin K, and different forms of vitamin E such as alpha, beta, gamma or delta tocopherols or their mixtures, and derivatives thereof.

Examples of other antioxidants include, but are not limited to, water-soluble antioxidants such as sulfhydryl compounds and their derivatives (e.g., N-acyl-cysteine), lipoic acid and dihydrolipoic acid, resveratrol, lactoferrin, and ascorbic acid derivatives (e.g., ethyl L-ascorbic acid, ascorbyl palmitate and ascorbyl polypeptide) and epigallocatechin gallate (EGCG). Oil-soluble antioxidants suitable for use in the compositions of this invention include, but are not limited to, butylated hydroxytoluene, retinoids (e.g., retinol and retinyl palmitate), tocopherols (e.g., tocopherol acetate), tocotrienols, and ubiquinone. Natural extracts containing antioxidants suitable for use in the compositions of this invention, include, but not limited to, extracts containing flavonoids and isoflavonoids and their derivatives (e.g., genistein and diadzein), extracts containing resveratrol and the like. Examples of such natural extracts include grape seed, pine bark, and propolis.

Preferably, the composition comprises another cosmetically acceptable active ingredient selected from hyaluronic acid, feverfew, Moringa Oleifera seed extract,

EGCG, N-acetyl glucosamine, gluconolactone, niacinamide and ethyl L-ascorbic acid.

A preferred composition comprises ascorbic acid, caffeine, sodium bisulfite and hyaluronic acid. Such a composition may comprise about 8 to about 12, for example about 10, % by weight of ascorbic acid, about 2% by weight of caffeine and about 0.05 % by weight of hyaluronic acid, all based on the total weight of the composition.

### Topical Compositions

The compositions of the present invention are applied topically to skin or hair, preferably skin. They are single phase aqueous compositions.

The compositions may be made into a wide variety of liquid product types that include but are not limited to solutions, lotions, creams, gels, sprays, ointments, and washes. The composition is preferably in the form of a serum, which is a liquid that is light and easily spread on and absorbed by the skin.

The composition may contain one or more emollients as known in the art. As used herein, “emollients” refer to materials used for the prevention or relief of dryness, such as by preventing the transepidermal loss of water from the skin. Examples of emollients include, but are not limited to, those set forth in the International Cosmetic Ingredient Dictionary and Handbook, eds. Pepe, Wenninger and McEwen, pp. 2930-36 (The Cosmetic, Toiletry, and Fragrance Assoc., Washington, D.C., 9th Edition, 2002) (hereinafter “ICI Handbook”). Examples of particularly suitable emollients include vegetable oils, mineral oils, fatty esters, and the like.

The composition can also be formulated as a gel (e.g., an aqueous, alcohol, alcohol/water, or oil gel using a suitable gelling agent(s)). Suitable gelling agents for aqueous and/or alcoholic gels include, but are not limited to, natural gums, acrylic acid and acrylate polymers and copolymers, and cellulose derivatives (e.g., hydroxymethyl cellulose and hydroxypropyl cellulose). Suitable gelling agents for oils (such as mineral oil) include, but are not limited to, hydrogenated butylene/ethylene/styrene copolymer and hydrogenated ethylene/propylene/styrene copolymer. Such gels typically contain between about 0.1% and 5%, by weight, of such gelling agents.

The compositions may contain, in addition to the above components, a wide variety of additional oil-soluble materials and/or water-soluble materials conventionally used in compositions for use on skin and hair, at their art-established levels. These include humectants, pH adjusters, fragrances, dyes, and preservatives (e.g., parabens).

The composition may comprise one or more oils. As used herein, the term “oil” means a hydrophobic material, for example hydrocarbon-based oils, silicones, fatty acid derivatives, glycerides, vegetable oils, vegetable oil derivatives, alkyl esters, wax esters, beeswax derivatives, sterols, and phospholipids.

Suitable hydrocarbon oils include petrolatum, mineral oil, micro-crystalline waxes, squalene and combinations thereof.

Silicone oils include dimethicone, dimethiconol, phenyl dimethicone and cyclic polysiloxanes and combinations thereof. Silicone oils having viscosities from about 0.5 to about 100,000 centistokes at 25° C. may also be useful in the composition.

Glycerides include castor oil, sunflower seed oil, coconut oil and derivatives, vegetable oils and derivatives, palm oil, jojoba oil, Shea butter, lanolin and combinations thereof.

Alkyl ester oils include, but are not limited to, isopropyl esters of fatty acids and esters of long chain fatty acids. More preferably, the following alkyl esters are useful: isopropyl palmitate, isopropyl myristate, myristyl myristate, isohexyl palmitate, decyl oleate, isononyl isononanoate and combinations thereof.

Preferably, the composition contains caprylyl glycol. More preferably the composition contains at least about 0.25% by weight of caprylyl glycol based on the total weight of the composition. For example, the composition may comprise at least about 0.5% by weight of caprylyl glycol based on the total weight of the composition.

Optionally, the composition may be substantially free of phenoxyethanol.

### pH

In one embodiment, the topical composition has a pH of about 3.5 to about 4. The composition may comprise a pH adjuster such as sodium hydroxide.

The topical composition may comprise a buffering agent such as lactic acid, citric acid, malic acid, tartaric acid, gluconic acid, or gluconolactone to maintain the pH. Typically, the composition contains about 2 to about 12, or about 4 to about 8, weight percent of a buffering agent.

### Method of Preparation

The compositions of the present invention may be prepared using methodology that is well known in the art. However, preferably the compositions are made by mixing the ingredients in a particular order to optimize their solubility in the composition. In particular, a composition according to the invention is preferably made by addition of caffeine to a water/inorganic acid mixture first, in order to dissolve the caffeine fully, followed by the addition of other ingredients (as desired) and ascorbic acid. Mixing is preferably done at an elevated temperature.

### Methods of Treating Skin

According to the invention, skin in need of treatment for skin brightening or lightening may be treated by topically applying a composition comprising about 5% to about 22% by weight of ascorbic acid based on the total weight of the composition, about 1 to about 5% by weight of caffeine based on the total weight of the composition, at least 0.2% by weight of an inorganic salt selected from the group consisting of sodium sulfite, potassium sulfite, sodium bisulfite, and sodium metabisulfite based on the total weight of the composition, and at least 28% by weight of water based on the total weight of the composition, wherein the composition is a single aqueous phase.

Also according to the invention, skin in need of treatment for skin brightening or lightening may be treated by topically applying a composition comprising about 10% by weight of ascorbic acid based on the total weight of the composition, about 2% by weight of caffeine based on the total weight of the composition, about 0.5% by weight of sodium bisulfite based on the total weight of the composition, at least 38% by weight of water based on the total weight of the composition, about 40% by weight 1,3-propanediol based on the total weight of the composition, and about 0.2% by weight of ethylenediaminetetraacetic acid based on the total weight of the composition, wherein the composition is a single aqueous phase.

Further according to the invention, skin in need of treatment for skin brightening or lightening may be treated by topically applying a composition comprising about 15% by weight of ascorbic acid based on the total weight of the composition, about 2% by weight of caffeine based on the total weight of the composition, about 0.5% by weight of sodium bisulfite based on the total weight of the composition, at least 33% by weight of water based on the total weight of the composition, about 45% by weight 1,3-propanediol based on the total weight of the composition, and about 0.2% by weight of ethylenediaminetetraacetic acid based on the total weight of the composition.

Further according to the invention, skin in need of treatment for skin brightening or lightening may be treated by topically applying a composition comprising about 20% by weight of ascorbic acid based on the total weight of the composition, about 2% by weight of caffeine based on the total weight of the composition, about 0.5% by weight of sodium bisulfite based on the total weight of the composition, at least 28% by weight of water based on the total weight of the composition, about 45% by weight 1,3-propanediol based on the total weight of the composition, and about 0.2% by weight of ethylenediaminetetraacetic acid based on the total weight of the composition.

Compositions according to the invention comprising ascorbic acid, caffeine, an inorganic salt, and water also advantageously and surprisingly provide superior ascorbic acid solubility and stability.

The following non-limiting examples further illustrate the invention.

### EXAMPLE 1

A series of compositions were made using the ingredients in Table 1.

TABLE 1

| | Composition Number | | | | | |
|---|---|---|---|---|---|---|
| INCI Names | 1 | 2 | 3 | 4 | 5 | 6 |
| Water | 39.45 | 39.35 | 39.35 | 39.25 | 38.45 | 44.65 |
| 1,3-Propanediol | 47 | 47 | 47 | 47 | 47 | 40 |
| Disodium EDTA | 0.2 | 0.2 | 0.2 | 0.2 | 0.2 | 0.2 |
| Sodium Bisulfite | 0 | 0 | 0 | 0.2 | 0 | 0.5 |
| Caffeine | 0 | 0 | 0 | 0 | 1 | 2 |
| Ethyl L-Ascorbic | 0 | 0.1 | 0 | 0 | 0 | 0 |
| Acid | | | | | | |
| EGCG | 0 | 0 | 0.1 | 0 | 0 | 0 |
| Caprylyl Glycol | 0.25 | 0.25 | 0.25 | 0.25 | 0.25 | 0.5 |
| Ascorbic Acid | 12.5 | 12.5 | 12.5 | 12.5 | 12.5 | 12.0 |
| Water; Glycerin; | 0.5 | 0.5 | 0.5 | 0.5 | 0.5 | 0 |
| Moringa Oleifera | | | | | | |
| Seed Extract | | | | | | |
| Sodium | 0.1 | 0.1 | 0.1 | 0.1 | 0.1 | 0.1 |
| Hydroxide | | | | | | |
| Hydrolyzed | 0 | 0 | 0 | 0 | 0 | 0.05 |
| Hyaluronic Acid | | | | | | |
| TOTAL | 100 | 100 | 100 | 100 | 100 | 100 |

The compositions were made as follows. The ingredients, when used, were added in the following order.
1. Add Water to the main beaker. Start mixing and heating to 50 C.
2. Add Hydrolyzed Hyaluronic Acid, mix until dissolved.
3. Add Disodium EDTA, mix until dissolved.
4. Add Sodium Bisulfite, mix until dissolved.
5. Add Caffeine, mix until dissolved.
6. Add Capryl Glycol, mix until dissolved.
7. Add Propanediol, mix until uniform.
8. Add Ascorbic Acid, mix until dissolved.
9. Start cooling the batch to 25 C.
10. Add Sodium hydroxide (20% NaOH Solution) to adjust final pH between 3.5 -4.0.

### EXAMPLE 2

The combined anti-oxidant effects of combinations of ascorbic acid with various other known anti-oxidants was tested using the oxygen radical absorbance capacity (ORAC) assay according to the method described by Ou B, Hampsch Woodill M, Prior R.L. Development and validation of an improved oxygen radical absorbance capacity assay using fluorescein as the fluorescent probe. J Agric Food Chem. 2001, 49:4619 (using a slightly different ex/em wavelength readout and a narrower range of Trolox standards). This assay measures the ability of anti-oxidant “chain breakers” to inhibit the decline in disodium fluorescein (FL) fluorescence that is induced by the peroxyl radical generator, 2′,2′-Azobis (2-amidinopropane) dihydrochloride (AAPH). Distilled water was used as the negative control. Trolox (6-hydroxy-2,5,7,8-tetramethylchroman-2-carboxylic acid, a water-soluble analog of vitamin E sold by Hoffman-LaRoche having antioxidant properties) was used as the positive control.

The final ORAC values were calculated from the regression equation of net areas under the FL decay curve using a formula where Area Under the Curve (AUC)=1+(Point 1+Point 2+Point 3+ . . . +Point 15)/Point 1. The Trolox standards were then plotted as micromoles over AUC. The samples were solved using their AUC yielding the values in micromole (μmol) Trolox equivalent (TE) per ml. Applied BioSystems Cytofluor 4000 fluorometer & software was used for quantification. ORAC values of the test materials were expressed in μmoles TE/ml (TE=Trolox Equivalents).

All ingredients were stored at room temperature and then diluted with distilled water into aqueous stock solutions for testing. Further dilutions were made in distilled water within 24 h from testing.

The results are shown in Tables 2 and 3, which report two separate test runs.

TABLE 2

| | | Expected | |
|---|---|---|---|
| | ORAC | (Additive) | |
| | (μmoles | ORAC | |
| Test Material | TE/ml) | value | Synergy |
| Water (Negative Control) | 0 | | |
| 10% ascorbic acid | 751 | | |
| 0.5% Aqua (and) Glycerin (and) | 12 | | |
| Avena Sativa (Oat) Kernel Extract | | | |
| 2% caffeine | 10 | | |
| 0.5% Ethyl L-Ascorbic Acid | 13 | | |
| 0.5% sodium ascorbyl phosphate | 10 | | |
| 0.5% Tetrahydrodiferuloyl-methane | 4 | | |
| 0.01% Mannitol (and) Ergothioneine | 0 | | |
| 0.1% Ergothioneine (and) Tricholoma | 0 | | |
| Matsutake Mycelium Ferment | | | |
| Extract) | | | |
| 0.5% Ergothioneine (and) Tricholoma | 1.5 | | |
| Matsutake Mycelium Ferment | | | |
| Extract) | | | |
| 0.5% Chrysanthemum Parthenium | 4 | | |
| (Feverfew) Extract | | | |
| 0.1% EGCG | 7 | | |
| 10% Vit C + 0.5% Aqua (and) | 556 | 763 | No |
| Glycerin (and) Avena Sativa | | | |
| (Oat) Kernel Extract | | | |
| 10% Vit C + 2% caffeine | 1111 | 761 | Yes |
| 10% Vit C + 0.5% Ethyl L-Ascorbic | 924 | 764 | Yes |
| Acid | | | |
| 10% Vit C + 0.5% sodium ascorbyl | 697 | 761 | No |
| phosphate | | | |
| 10% Vit C + 0.5% | 263 | 755 | No |
| Tetrahydrodiferuloyl-methane | | | |
| 10% Vit C + 0.01% Mannitol (and) | 628 | 751 | No |
| Ergothioneine | | | |
| 10% Vit C + 0.1% Ergothioneine | 602 | 751 | No |
| (and) Tricholoma Matsutake | | | |
| Mycelium Ferment Extract) | | | |
| 10% Vit C + 0.5% Ergothioneine | 598 | 752.2 | No |
| (and) Tricholoma Matsutake | | | |
| Mycelium Ferment Extract) | | | |
| 10% Vit C + 0.5% Chrysanthemum | 638 | 755 | No |
| Parthenium (Feverfew) Extract | | | |
| 10% Vit C + 0.1% EGCG | 859 | 758 | Yes |
| Trolox (Positive Control) | 3940 μmol/g | | |

TABLE 3

| | ORAC | Expected | |
|---|---|---|---|
| | (μmoles | (Additive) | |
| Test Material | TE/ml) | ORAC value | Synergy |
| Water (Negative Control) | 0 | | |
| 10% ascorbic acid | 838 | | |
| 1% caffeine | 0 | | |
| 0.1% Ethyl L-Ascorbic Acid | 7 | | |
| 10% Vit C + 1% Caffeine | 688 | 838 | No |
| 10% Vit C + 0.1% Ethyl | 891 | 845 | Yes |
| L-Ascorbic Acid | | | |
| Trolox (Positive Control) | 4368 μmol/g | | |

The combination of ascorbic acid with 2% caffeine was one of only four combinations that provided a synergistic ORAC value. The expected (additive) ORAC value for the combination of ascorbic acid with 2% caffeine was 761 TE per ml. However, the combination surprisingly provided an ORAC value of 1111 TE per ml. This combination increased the vitamin C's antioxidant activity by 48%.

### EXAMPLE 3

The stability of ascorbic acid in a composition according to the invention comprising water, ascorbic acid, caffeine, sodium bisulfite and EDTA (Composition 7) was compared with the stability of ascorbic acid in a comparative composition comprising water and ascorbic acid but no caffeine, sodium bisulfite, or EDTA (Composition 8). The ingredients in Compositions 7 and 8 are shown in Table 4. On preparation, both compositions contained 12 wt % ascorbic acid.

TABLE 4

| | Composition 7 | Composition 8 |
|---|---|---|
| US INCI | % wt | % wt |
| Water | 39.05 | 42.25 |
| Hydrolyzed Hyaluronic | 0.1 | 0.1 |
| Acid | | |
| Disodium EDTA | 0.2 | 0 |
| Sodium Bisulfite | 1 | 0 |
| Caffeine | 2 | 0 |
| Caprylyl Glycol | 0.5 | 0.5 |
| Propanediol | 45 | 45 |
| Ascorbic Acid | 12 | 12 |
| Sodium Hydroxide | 0.15 | 0.15 |
| Total: | 100 | 100 |

Ascorbic acid stability in the compositions was determined using high pressure liquid chromatography (HPLC) to detect the content of ascorbic acid over time. Samples were dispersed and dissolved in mobile phase, mixed, and filtered prior to injection onto a HYPERSIL ODS C18, 250×4.6 mm column, detection at 210 nm. The testing times and conditions were as follows:
Room Temperature (1 Month, 2 Months and 3 Months);
40° C. (1 month, 2 Months and 3 Months); and
50° C. (1 Week and 2 Weeks).

The results are shown in Table 5, which shows the weight percent of ascorbic acid remaining in the two compositions over time.

TABLE 5

| | Composition 7 | Composition 8 |
|---|---|---|
| CONDITION | % wt | % wt |
| 1 month at room temp. | 11.682 | 11.8607 |
| 2 months at room temp. | 11.559 | 11.578 |
| 3 months at room temp. | 11.831 | 11.506 |
| 1 month at 40° C. | 11.129 | 11.0757 |
| 2 months at 40° C. | 10.511 | 9.608 |
| 3 months at 40° C. | 9.772 | 8.842 |
| 1 week at 50° C. | 11.259 | 11.183 |
| 2 weeks at 50° C. | 10.45 | 10.603 |

Composition 7 according to the invention maintained good ascorbic acid stability at all time points and conditions. In contrast, the amount of ascorbic acid in comparative Composition 8 at 40° C. was decreased relative to the amount in Composition 7. In particular, the amount of ascorbic acid in comparative Composition 8 at 40° C. after 2 months and 3 months was significantly lower than that in Composition 7 at the same time points.

1. A topical composition comprising about 5% to about 22% by weight of ascorbic acid based on the total weight of the composition, about 1 to about 5% by weight of caffeine based on the total weight of the composition, at least 0.2% by weight of an inorganic salt selected from the group consisting of sodium sulfite, potassium sulfite, sodium bisulfite, and sodium metabisulfite based on the total weight of the composition, and at least 28% by weight of water based on the total weight of the composition, wherein the composition is a single aqueous phase.

2. The composition of claim 1 further comprising at least 35% by weight of 1,3-propanediol based on the total weight of the composition.

3. The composition of claim 1 further comprising at least 0.1% by weight of ethylenediaminetetraacetic acid based on the total weight of the composition.

4. The composition of claim 1, wherein the inorganic salt is sodium bisulfite.

5. The composition of claim 1 further comprising hyaluronic acid.

6. A topical composition comprising about 10% by weight of ascorbic acid based on the total weight of the composition, about 2% by weight of caffeine based on the total weight of the composition, about 0.5% by weight of sodium bisulfite based on the total weight of the composition, at least 38% by weight of water based on the total weight of the composition, about 40% by weight 1,3-propanediol based on the total weight of the composition, and about 0.2% by weight of ethylenediaminetetraacetic acid based on the total weight of the composition, wherein the composition is a single aqueous phase.

7. A topical composition comprising about 15% by weight of ascorbic acid based on the total weight of the composition, about 2% by weight of caffeine based on the total weight of the composition, about 0.5% by weight of sodium bisulfite based on the total weight of the composition, at least 33% by weight of water based on the total weight of the composition, about 45% by weight 1,3-propanediol based on the total weight of the composition, and about 0.2% by weight of ethylenediaminetetraacetic acid based on the total weight of the composition.

8. A topical composition comprising about 20% by weight of ascorbic acid based on the total weight of the composition, about 2% by weight of caffeine based on the total weight of the composition, about 0.5% by weight of sodium bisulfite based on the total weight of the composition, at least 28% by weight of water based on the total weight of the composition, about 45% by weight 1,3-propanediol based on the total weight of the composition, and about 0.2% by weight of ethylenediaminetetraacetic acid based on the total weight of the composition.

9. A cosmetic method for treating skin, comprising topically applying to skin in need of treatment for skin brightening or lightening the topical composition of claim 1.
